(19) United States
(12) Patent Application Publication
(10) Pub. No.: US 2009/0012189 A1
(43) Pub. Date: Jan. 8, 2009

(54) Catalyst Bodies for Use in Fischer-Tropsch Reactions

(76) Inventors: Arold Marcel Albert Routier, Amsterdam (NL); Franciscus Johannes Maria Schrauwen, Amsterdam (NL); Gerardus Petrus Niesen, Amsterdam (NL)

Correspondence Address: SHELL OIL COMPANY, P O BOX 2463, HOUSTON, TX, 772522463, US

(21) Appl. No.: 12/097,267

(22) PCT Filed: Dec. 14, 2006
(86) PCT No.: PCT/EP2006/069695

§ 371 Date: Jun. 13, 2008

(30) Foreign Application Priority Data
Dec. 16, 2005 (EP) 05112320.6

(51) Int. Cl. C07C 1/04; B01J 35/04

(52) U.S. Cl. 518/715; 502/4; 518/728

(57) ABSTRACT

The invention relates to a catalyst body comprising a Fischer-Tropsch catalyst or catalyst precursor and a porous body, said porous body being between 1-50 mm, preferably 1-30 mm in size, the catalyst body having an internal voidage between 50-95%. The invention further relates to a process comprising the steps of: (i) introducing the synthesis gas into the reactor; and (ii) contacting the synthesis gas with a non-stationary catalyst to catalytically convert the synthesis gas at an elevated temperature to obtain the normally gaseous, normally liquid, and optionally normally solid hydrocarbons from synthesis gas; wherein the catalyst of step (ii) is located on a plurality of porous bodies being 1-50 mm in size, preferably 1-30 mm in size, thus forming catalyst bodies, and wherein said catalyst bodies have an external voidage in situ in the reactor between 5-60%, and a porosity within the catalyst bodies between 50-95%. Use of catalyst bodies according to the invention provides an advantageous intermediate balance whereby such catalyst bodies are significantly easier (and therefore less costly) to separate from the products of the slurry reactor, but they are still able to be supported by the slurry, and are therefore still movable within the reactor vessel so as to seek the most even catalytic transfer and heat transfer, but without being fixed.

The present invention relates to catalyst bodies for use in slurry reactors. In particular, the invention relates to catalyst bodies for use in Fischer-Tropsch reactions.

The Fischer-Tropsch process can be used for the conversion of hydrocarbonaceous feed stocks into liquid and/or solid hydrocarbons. The feed stock (e.g. natural gas, associated gas and/or coal-bed methane, coal) is converted in a first step into a mixture of hydrogen and carbon monoxide (this mixture is often referred to as synthesis gas or syngas). The synthesis gas is then converted in one or more steps over a suitable catalyst at elevated temperature and pressure into paraffinic compounds ranging from methane to high molecular weight molecules comprising up to 200 carbon atoms, or, under particular circumstances, even more.

Numerous types of reactor systems have been developed for carrying out the Fischer-Tropsch reaction. For example, Fischer-Tropsch reactor systems include fixed bed reactors, especially multi tubular fixed bed reactors, fluidised bed reactors, such as entrained fluidised bed reactors and fixed fluidised bed reactors, and slurry bed reactors such as three-phase slurry bubble columns and ebullated bed reactors.

The Fischer-Tropsch reaction is very exothermic and temperature sensitive with the result that careful temperature control is required to maintain optimum operation conditions and desired hydrocarbon product selectivity. Bearing in mind the very high heat of reaction which characterises the Fischer-Tropsch reaction, the heat transfer characteristics and cooling mechanisms of a reactor are very important.

Three-phase slurry bubble column reactors potentially offer advantages over the fixed-bed design in terms of heat transfer performance. Such reactors typically incorporate small catalyst particles in a liquid continuous matrix. The synthesis gas is bubbled through, maintaining suspension of small catalyst particles and providing the reactants. In the case of multi-tubular reactors, the number of tubes incorporated is generally limited by mechanical parameters. The motion of the continuous liquid matrix promotes heat transfer to achieve a high commercial productivity. The catalyst particles are moving within a liquid continuous phase, resulting in efficient transfer of heat generated by the catalyst particles to the cooling surfaces. The large liquid inventory in the reactor provides a high thermal inertia, which helps prevent rapid temperature increases that can lead to thermal runaway.

The micron-sized catalyst particles must be removed from the reaction products, as at least part of the reaction products are in the liquid phase under reactor conditions. Because of the small size of the particles this separation is difficult, and is typically carried out using expensive internal or external filtration system. Other issues associated with the use of suspended catalyst particles are non-uniform distribution of catalyst throughout the reactor (with knock-on effects on cooling) and catalyst attrition.

U.S. Pat. No. 6,262,131B1 discloses a “structural” Fischer-Tropsch catalyst disposed in a reactor with a certain voidage ratio, generally being a truncated or fragmented randomly packed arrangement. The structural catalyst however is still used in a stationery (i.e. no large-scale movement) packed bed.

U.S. Pat. No. 6,211,255B1 discloses a monolithic catalyst. The monolith has channels, but could still prevent random and turbulent flow generally desired in a Fischer-Tropsch reactor to ensure good mixing of the reactants.

It is one object of the present invention to provide catalyst bodies for use in Fischer-Tropsch slurry reactors which addresses some of the disadvantages described above in relation to catalysts employed in known reactor set-ups.

Further objects of the invention will be apparent from the detailed description that follows.

Accordingly, the present invention provides a process for producing normally gaseous, normally liquid, and optionally normally solid hydrocarbons from synthesis gas in a three-phase reactor comprising the steps of:

(i) introducing the synthesis gas into the reactor; and
(ii) contacting the synthesis gas with a non-stationary catalyst to catalytically convert the synthesis gas at an elevated temperature to obtain the normally gaseous, normally liquid, and optionally normally solid hydrocarbons from synthesis gas;
wherein the catalyst of step (ii) is located on a plurality of porous bodies being 1-50 mm in size.

The porous bodies act as support for the catalyst that is located on these bodies. Porous bodies on which a catalyst or catalyst precursor is present will be referred to as “catalyst bodies”.

In a preferred embodiment said porous bodies are 1-30 mm in size.

When preparing catalyst bodies suitable for use in a process according to the present invention, preferably use is made of porous bodies of which more than 95%, more preferably more than 99%, most preferably 100%, has a size of 1-50 mm, preferably 1-30 mm.

The porous bodies may be of regular or irregular shapes, or a mixture thereof. Such include cylinders, cubes, spheres, ovoids, etc, and other shaped polygons. In general, “size” can be considered as their longest internal straight length.

In a preferred embodiment the porous bodies have a form or shape selected from the group consisting of gauze, honeycomb, monolith, sponge, mesh, webbing, foil construct and woven mat form, or any combination of these.

Obviously, the porous bodies may be a combination of forms such as those listed above. For example, the porous bodies may be made up of honeycomb shaped material and have a circular outer shape. Another example is a cylinder made from woven mat.

The porous bodies may suitably be made from refractory oxides; for example titania (TiO₂), silica (SiO₂), alumina; metals, for example stainless steel, iron or copper; or any similar inert material capable of withstanding conditions within the reactor.

The external voidage of the catalyst bodies, i.e. the porous bodies on which the catalyst has been applied, in situ in the reactor is between 5-60%, preferably less than 40% by volume, more preferably about 20% by volume.

The porosity within the catalyst bodies, i.e. the internal voidage of the catalyst bodies, is within the range 50-95%; preferably the internal voidage is more than 60%, more preferably more than 70%, even more preferably more than 80%, and most preferably more than 90% (with reference to the circumferential volume of the bodies). Before application of the catalyst on the porous bodies, the porosity within the porous bodies may be up to 98%.

The open volume within the catalyst bodies must be sufficient to facilitate efficient through-flow of reactants, while at the same time the specific surface area of each catalyst body should be as large as possible to increase exposure of reactants to the catalyst material. The open nature of the catalyst bodies of the present invention also allow the same or similar catalyst loading to be achieved as with prior micron-sized catalyst particles, such there is no reduction of the catalyst activity and STY by use of bigger catalyst bodies.

Suitable porous bodies, on which the catalyst can be applied, can be prepared in-house or alternatively be obtained commercially. An example of a producer of suitable porous bodies is the Fraunhofer-Institute for Manufacturing and Advanced Materials in Dresden, Germany.

The Fraunhofer-Institute advertises and sells, for example, melt extracted metallic fibres, and highly porous fibre structures that can be cylindrically or spherically shaped.

The catalyst is generally formed from a catalyst precursor material. More preferably, each porous catalyst body comprises a Fischer-Tropsch catalyst material.

The normally gaseous, normally liquid, and normally solid hydrocarbons in which the synthesis gas is to be converted are hydrocarbons that are respectively gaseous, liquid and solid at room temperature at about 1 atm.

The present invention fits between the use of the small catalyst particles in typical prior art slurry reactors, which are 5-150 μm in size (and which are therefore difficult to separate from the slurry), and the use of much larger and immobilised catalyst structures such as those in U.S. Pat. No. 6,262,131B1, which can be difficult to load in the reactor, and which lead to uneven catalyst dispersion, and so uneven activity and ‘hot spots’. Use of catalyst bodies comprising porous bodies with a minimum size of 1 mm, and a maximum size of up to 50 mm, provides an advantageous intermediate balance whereby such catalyst bodies are significantly easier (and therefore less costly) to separate from the products of the slurry reactor, but they are still able to be supported by the slurry, and are therefore still movable within the reactor vessel so as to seek the most even catalytic transfer and heat transfer, but without being fixed.

In particular, the present invention circumvents the disadvantages of fixed bed multi-tubular reactor technology (such as limited catalyst utilisation due to mass transport limitation within catalyst particles, heat removal limited by transport through the catalyst bed, and the expense of this type of reactor and high pressure drop) and the disadvantages of current slurry reactor technology such as non-uniform axial catalyst hold-up, catalyst attrition, the need for expensive filtration means for separation of small catalyst particles from the product wax, and catalyst entrainment.

Thus, the present invention also provides a catalyst body including a catalyst or catalyst precursor, preferably a Fischer-Tropsch catalyst or catalyst precursor, and a porous body having a size of 1-50 mm, preferably 1-30 mm. The porous body is able to support a catalyst or catalyst precursor for use in a hydrocarbon synthesis reactor. The catalyst body is not fixed within the reactor.

Because the catalyst bodies of the present invention are moving in use, the mass transport limitation of the syngas components is negligible.

Preferably the catalyst or catalyst material, or a precursor thereof, is applied as a layer to the porous bodies, typically in a thickness of from about 1 to about 300 microns and preferably from about 5 to about 200 microns.

It is preferred that the catalyst fraction of the catalyst bodies is at least about 1% by volume and preferably greater than about 4% by volume (with reference to the volume of the catalyst bodies), with a preferred maximum of 25% by volume.

General methods of preparing catalyst and catalyst materials and forming catalyst mixtures are known in the art, see for example U.S. Pat. No. 4,409,131, U.S. Pat. No. 5,783,607, U.S. Pat. No. 5,502,019, WO 0176734, CA 1166655, U.S. Pat. No. 5,863,856 and U.S. Pat. No. 5,783,604. These include preparation by co-precipitation and impregnation. Such processes could also include freezing, sudden temperature changing, etc. Control of the component ratio in the solid solution can be provided by parameters such as residence time, temperature control, concentration of each component, etc.

The catalyst material, generally based on a catalytically active metal, may be present with one or more metals or metal oxides as promoters, more particularly one or more d-metals or d-metal oxides.

Suitable metal oxide promoters may be selected from Groups 2-7 of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are most suitable promoters.

Suitable metal promoters may be selected from Groups 7-10 of the Periodic Table. Manganese, iron, rhenium and Group 8-10 noble metals are particularly suitable, with platinum and palladium being especially preferred. The amount of promoter present in the catalyst is suitably in the range of from 0.01 to 100 pbw, preferably 0.1 to 40, more preferably 1 to 20 pbw, per 100 pbw of carrier.

References to “Groups” and the Periodic Table as used herein relate to the new IUPAC version of the Periodic Table of Elements such as that described in the 87^{th} Edition of the Handbook of Chemistry and Physics (CRC Press).

The catalyst material could also be present with one or more co-catalysts. Suitable co-catalysts include one or more metals such as iron, nickel, or one or more noble metals from Groups 8-10. Preferred noble metals are platinum, palladium, rhodium, ruthenium, iridium and osmium. Most preferred co-catalysts for use in the hydro-cracking are those comprising platinum. Such co-catalysts are usually present in small amounts.

A suitable catalyst comprises cobalt as the catalytically active metal and zirconium as a promoter. Another suitable catalyst comprises cobalt as the catalytically active metal and manganese and/or vanadium as a promoter.

The catalyst material preferably also includes a support or carrier, such as a porous inorganic refactory oxide, such as alumina, silica, titania, zirconia or mixtures thereof. Most preferably, the carrier material is titania. The carrier could be added onto the porous bodies of the present invention prior to addition of the catalytically active metal by impregnation for example. Alternatively, the catalytically active metal and carrier material could be admixed and then added to the porous bodies of the present invention. For example, a powder form of the catalyst material could be formed into a slurry, and then spray coated onto the porous bodies.

Any promoter(s) are typically present in an amount of from 0.1 to 60 parts by weight per 100 parts by weight of a porous carrier. It will however be appreciated that the optimum amount of promoter(s) may vary for the respective elements which act as promoter(s). If the catalyst comprises cobalt as the catalytically active metal and manganese and/or vanadium as promoter, the cobalt: (manganese+vanadium) atomic ratio is advantageously between 5:1-30:1.

In one embodiment of the present invention, the catalyst comprises the promoter(s) and/or co-catalyst(s) having a concentration in the Group 8-10 metal(s) in the range 1-10 atom %, preferably 3-7 atom %, and more preferably 4-6 atom %.

Preferably the synthesis gas is hydrogen and carbon monoxide, typically fed into the slurry reactor at a molar ratio in the range of from 0.4 to 2.5 and preferably at a molar ratio of 1.0 to 2.5.

The present invention also provides a process further comprising:

(iii) catalytically hydrocracking higher boiling range paraffinic hydrocarbons produced in step (ii),

as well as hydrocarbons whenever provided by a process as described herein.

The present invention also provides use of catalyst bodies as defined herein in a process for producing normally gaseous, normally liquid and optionally normally solid hydrocarbons from synthesis gas which comprises the steps of:

(i) providing the synthesis gas; and

(ii) catalytically converting the synthesis gas of step (i) at an elevated temperature and pressure to obtain the normally gaseous, normally liquid and optionally normally solid hydrocarbons.

The catalyst bodies of the present invention are suitable for slurry reactions, such as for example Fischer-Tropsch type reactions. Suitable slurry liquids are known to those skilled in the art.

Typically, at least a part of the slurry liquid is a reaction product of the exothermic reaction. The reaction mixture typically comprises synthesis gas and hydrocarbon feedstock reactants and liquid hydrocarbon products.

The catalyst material may for example be a heavy paraffin synthesis catalyst such as those known to the person skilled in the art. Several suitable catalyst materials are exemplified below. The catalyst material is applied to the porous bodies as a thin layer. The catalyst layer should be sufficiently thin to avoid diffusional mass transport limitation (decrease of CO and/or hydrogen partial pressure and/or unfavourable change of the hydrogen/carbon monoxide-ratio within the catalyst layer) of the syngas components within the catalyst layer. Thickness of the catalyst layer can be increased up to the onset of mass transport limitation. There is no upper limit to the thickness of the catalyst layer onto the porous bodies other than mass transport limitation and voidage of the substrate for hydrodynamic reasons. This provides additional freedom in comparison to a slurry reactor, where the size/density of the slurry catalyst particles imposes an upper limit to the size (with too high a settling velocity resulting from a particle size which is too large and which causes non-uniform catalyst hold-up along the height of the reactor).

In relation to the geometry of the catalyst bodies, it is desirable to have gas/liquid hydrodynamics such that the high heat transfer coefficients from process side to cooling surface of a gas-liquid bubble column or three-phase gas/liquid/suspended catalyst system, are retained or at least approached. Liquid mixing may be enhanced by the structure of the catalyst bodies.

Liquid movement and mixing within the reactor is an important aspect of the invention. The liquid product wax provides bulk transport of syngas components to the catalyst surface. The liquid phase is also the main carrier of generated process heat from the catalyst to the cooling means. Mixing of the liquid phase and movement of the liquid along the walls of the cooling elements may be at least partly generated by the gas (syngas in addition to light hydrocarbon products in the vapour phase) rising through the liquid filled voids in the catalyst body structure.

In relation to the geometry of the catalyst bodies, it is also desirable to have gas/liquid hydrodynamics such that high mass transfer coefficients for transfer of the hydrogen and carbon monoxide of the synthesis gas from the gas phase to the liquid phase are achieved. A reactor containing the catalyst bodies of the invention can be configured such that there are no significant gas-to-liquid or liquid-to-gas mass transport limitations during operation. The combination of gas distribution, gas-liquid interfacial area, mass transfer coefficients and liquid mixing should ensure near saturation of the bulk liquid with syngas anywhere within the catalyst bodies. The gas also serves as the main driver for liquid convection and mixing, ensuring effective transport of heat through the cooling means and thus a uniform temperature profile. Liquid convection could even be optimised within the dimensional scale of the porous nature of the catalyst bodies.

The process of the present invention is preferably a reaction which is carried out in the presence of a solid catalyst. Typically, at least one of the reactants of the exothermic reaction is gaseous. Examples of exothermic reactions include hydrogenation reactions, hydroformylation, alkanol synthesis, the preparation of aromatic urethanes using carbon monoxide, Kölbel-Engelhardt synthesis, polyolefin synthesis, and Fischer-Tropsch synthesis. According to a preferred embodiment of the present invention, an exothermic reaction is a Fischer-Tropsch synthesis reaction.

One process for producing normally gaseous, normally liquid, and optionally normally solid hydrocarbons from synthesis gas in a three-phase reactor is the Fischer-Tropsch synthesis reaction.

The Fischer-Tropsch synthesis is well known to those skilled in the art and involves synthesis of hydrocarbons from a gaseous mixture of hydrogen and carbon monoxide, by contacting that mixture at reaction conditions with a Fischer-Tropsch catalyst. Suitable slurry liquids are known to those skilled in the art. Typically, at least a part of the slurry liquid is a reaction product of the exothermic reaction. Preferably, the slurry liquid is substantially completely a reaction product (or products).

Examples of products of the Fischer-Tropsch synthesis (for a low temperature Co based system) may range from methane to heavy paraffinic waxes. Preferably in the case of a Co based catalyst, the production of methane is minimised and a substantial portion of the hydrocarbons produced have a carbon chain length of at least 5 carbon atoms. Preferably, the amount of C₅+ hydrocarbons is at least 60% by weight of the total product, more preferably at least 70% by weight, even more preferably at least 80% by weight, most preferably at least 85% by weight, of the total weight of hydrocarbonaceous products formed.

Fischer-Tropsch catalysts are known in the art, and typically include a Group 8-10 metal component, preferably cobalt, iron and/or ruthenium, more preferably cobalt. Typically the porous catalyst bodies comprise a carrier material such as a porous inorganic refractory oxide, preferably alumina, silica, titania, zirconia or mixtures thereof.

A most suitable catalyst material comprises cobalt and zirconium as a promoter. Another most suitable catalyst comprises cobalt and manganese and/or vanadium as a promoter.

The Fischer-Tropsch synthesis is preferably carried out at a temperature in the range from 125 to 350° C., more preferably 175 to 275° C., most preferably 180 to 260° C. The pressure preferably ranges from 5 to 150 bar abs., more preferably from 5 to 80 bar abs.

The gaseous hourly space velocity, may vary within wide ranges and is typically in the range from 500 to 20,000 NI/l/h preferably in the range from 700 to 10,000 NI/l/h (with reference to the volume of porous catalyst elements and the spaces therein between).

It will be understood that the skilled person is capable of selecting the most appropriate conditions for a specific reactor configuration and reaction regime.

1. A catalyst body comprising a Fischer-Tropsch catalyst or catalyst precursor and a porous body, said porous body being between 1-50 mm, preferably 1-30 mm in size, the catalyst body having an internal voidage between 50-95%.

2. A catalyst body according to claim 1 wherein the porous body has a gauze, honeycomb, monolith, mesh, webbing, sponge, foil construct or woven mat form.

3. A catalyst body according to claim 1 or 2 wherein the porous body is formed from a material selected from the group consisting of refractory oxides, metals, or mixtures thereof.

4. A catalyst body according to claim 3 wherein the porous body is formed from a refractory oxide material selected from the group consisting of titania, silica, zirconia, alumina and mixtures thereof.

5. A catalyst body according to claim 3 wherein the porous body is formed from stainless steel.

6. A catalyst body according to any one of claims 1 to 5 wherein the open volume within the catalyst body is more than 60%, preferably more than 70%, more preferably more than 80%.

7. A catalyst body according to any one of claims 1 to 6 wherein the catalyst or catalyst precursor is located as a layer upon the porous body, said layer preferably having an average thickness of from about 1 to 300 microns, preferably about 5 to about 200 microns.

8. A catalyst body according to any one of claims 1 to 7 wherein the catalyst fraction of the catalyst body is at least about 1% by volume, preferably at least about 4% by volume, with reference to the volume of the catalyst body.

9. A process for producing normally gaseous, normally liquid, and optionally normally solid hydrocarbons from synthesis gas in a three-phase reactor comprising the steps of:
(i) introducing the synthesis gas into the reactor; and
(ii) contacting the synthesis gas with a non-stationary catalyst to catalytically convert the synthesis gas at an elevated temperature to obtain the normally gaseous, normally liquid, and optionally normally solid hydrocarbons from synthesis gas;
wherein the catalyst of step (ii) is located on a plurality of porous bodies being 1-50 mm in size, preferably 1-30 mm in size, thus forming catalyst bodies, and
wherein said catalyst bodies have an external voidage in situ in the reactor between 5-60%, and a porosity within the catalyst bodies between 50-95%.

10. A process according to claim 9 wherein the catalyst bodies are a catalyst bodies according to any one or more of claims 1 to 8.

11. A process according to claim 9 or 10 wherein the catalyst of step (ii) is located on a plurality of porous bodies as a layer upon the porous bodies.

12. A process according to claim 11 wherein the catalyst layer on the porous bodies has an average thickness of from about 1 to 300 microns, preferably about 5 to about 200 microns.

13. A process according to any one of claims 9 to 12 wherein the active component of the catalyst is selected from the group consisting of cobalt, iron, ruthenium and mixtures thereof, preferably cobalt.

14. A process according to any one of claims 9 to 13 wherein step (ii) further comprises the use of a promoter.

15. A process according to claim 14 wherein the promoter is selected from the group consisting of zirconium, manganese, vanadium, rhenium, platinum, palladium and mixtures thereof, preferably manganese, vanadium and mixtures thereof.

16. A process for the synthesis of hydrocarbons according to any one of claims 9 to 15 further comprising a hydrocracking and/or hydro-treatment step.

17. Hydrocarbons whenever prepared by a process as claimed in any one of claims 9 to 16.

18. Use of catalyst bodies as defined in any one of claims 1 to 8 in a process for producing normally gaseous, normally liquid and optionally normally solid hydrocarbons from synthesis gas which comprises the steps of:
(i) providing the synthesis gas; and
(ii) contacting the synthesis gas with the catalyst material to catalytically convert the synthesis gas of step (i) at an elevated temperature and pressure to obtain the normally gaseous, normally liquid and optionally normally solid hydrocarbons.
